(19) United States
(12) Patent Application Publication
(10) Pub. No.: US 2026/0123900 A1
(43) Pub. Date: May 7, 2026

(54) BEDSIDE X-RAY DETECTOR DEPLOYMENT SYSTEM

(71) Applicant: HealthTech Strategies, LLC, Philadelphia, PA (US)

(72) Inventor: Luca Bogoni, Philadelphia, PA (US)

(21) Appl. No.: 19/379,558

(22) Filed: Nov. 4, 2025

Related U.S. Application Data
(60) Provisional application No. 63/715,716, filed on Nov. 4, 2024.

(51) Int. Cl. A61B 6/00; A61B 6/42; A61B 6/58

(52) U.S. Cl. CPC A61B 6/4405; A61B 6/42; A61B 6/587

(57) ABSTRACT

A bedside X-ray imaging system includes a mobile platform independent of a patient bed. A wheeled base with locking stabilizes the platform adjacent to the bed. A vertical support provides height alignment to a patient support surface. A conveyor belt deploys a detector housing carrying an X-ray detector from a retracted to an extended position beneath the patient. Deployment depth is indicated by markings on the belt or by a panel that reports detector position. A controller actuates the conveyor to place and retract the housing. The system can establish a calibrated coordinate relationship with an external X-ray source and provide planar in-housing X-Y displacement for fine positioning and continuous tracking during procedures.

### CROSS-REFERENCE TO RELATED APPLICATIONS

This application claims priority to U.S. Provisional Ser. No. 63/715,716, filed Nov. 4, 2024, the entire disclosure of which is incorporated herein by reference.

### FIELD

This disclosure relates to medical imaging devices and systems. More specifically, the disclosure relates to mobile bedside X-ray detector deployment systems that enable automated positioning of X-ray detectors beneath bedridden patients without requiring manual patient lifting or integration into patient support structures.

### BACKGROUND

X-ray imaging is a cornerstone of diagnostics in healthcare, particularly for critically ill, bedridden, ICU, or incapacitated patients. Imaging these patients typically requires substantial manual effort, with multiple healthcare workers needed to lift and position X-ray detectors beneath them. Once positioned, it is often impractical to reposition the detector for interventional procedures at the bedside.

Traditional bedside X-ray solutions lack the capacity for dynamic adjustments essential in interventional radiology for guiding tools like catheters. While some interventional procedures may be performed bedside after positioning the detector, the absence of continuous tracking limits the effectiveness of image-guided interventions, rendering many procedures feasible only in radiology suites. Transporting critically ill patients to these suites introduces considerable risks, including hemodynamic instability, respiratory compromise, line dislodgement, and the need for specialized monitoring, complicating care and increasing patient vulnerability.

The manual approach to placing detectors under bedridden patients presents several challenges. The typical process requires two to three healthcare workers, adding strain to healthcare facilities with limited staff availability. Lifting and positioning patients to insert detectors puts healthcare staff at risk of musculoskeletal disorders, with a significant portion reporting recurring back pain. These issues can lead to staff requiring disability leave, increasing financial burdens on healthcare institutions and contributing to burnout among care providers.

Detector placement and removal are cumbersome and time-consuming, delaying imaging procedures and possibly impacting patient throughput. Achieving optimal alignment between the X-ray source and detector is challenging in a manual setup, often resulting in misalignment that reduces image quality, necessitates retakes, and increases radiation exposure for patients. Patient manipulation for detector placement is invasive, uncomfortable, and can cause anxiety or distress, especially for critically ill patients.

Existing detector positioning systems include fixed bed-integrated architectures such as under-table cassette movers requiring integration into patient support apparatus, floating cabinet architectures such as table-integrated detector housings with multi-dimensional movement capabilities, and patient-elevating lift devices such as hydraulic systems requiring pre-positioning. These systems require either permanent integration into patient beds or table structures, or require manual pre-positioning and patient elevation, and do not provide mobile bedside deployment capabilities suitable for standard hospital beds in intensive care or general ward environments.

These limitations underscore the need for an automated, bedside X-ray solution that minimizes manual intervention, enhances image quality, and supports real-time positioning for interventional procedures, ensuring safer, more efficient, and patient-centered imaging for bedridden patients.

### SUMMARY

The present disclosure provides a bedside X-ray imaging system comprising a mobile platform, a vertical support structure, a conveyor belt mechanism with integrated position indicators, and associated control systems that enable automated deployment of an X-ray detector beneath a patient positioned on a patient bed, wherein the system is structurally independent of the patient bed and operates from a bedside position.

In one aspect, the bedside X-ray imaging system comprises a mobile platform comprising a wheeled base and a locking mechanism, wherein the locking mechanism stabilizes the mobile platform adjacent to a patient bed and wherein the mobile platform is structurally independent of the patient bed. A vertical support structure is coupled to the mobile platform, the vertical support structure comprising a height adjustment mechanism that positions a distal portion of the vertical support structure at a height corresponding to a patient support surface of the patient bed.

A conveyor belt mechanism is coupled to the vertical support structure and configured to extend from the vertical support structure in an orientation aligned and parallel to the patient support surface. The conveyor belt mechanism comprises a belt transport system that translates longitudinally relative to the vertical support structure to deploy from a retracted position to an extended position, a detector housing integrated with the belt transport system, wherein the detector housing travels with the belt transport system during longitudinal translation and houses an X-ray detector, and a position indicator that displays a deployment depth of the detector housing. The position indicator comprises at least one of visible markings on the belt transport system indicating longitudinal displacement or a display panel presenting a representation of detector housing position.

A controller is operatively coupled to the conveyor belt mechanism, wherein the controller actuates the belt transport system to deploy the detector housing from the retracted position to the extended position beneath a patient positioned on the patient support surface.

In various embodiments, the system further comprises communication interfaces for establishing calibrated coordinate systems with external X-ray sources, planar displacement mechanisms for fine positioning after deployment, proximity sensors for bed contact detection, and advanced interventional radiology tracking capabilities.

### BRIEF DESCRIPTION OF THE DRAWINGS

The accompanying drawings illustrate certain embodiments of the invention and are not to be considered limiting in scope:

FIGS. 1A and 1B illustrate the bedside X-ray imaging system. FIG. 1A is a perspective view of the mobile platform, vertical support structures, and key components. FIG. 1B is a block/internal view showing controller subsystems and internal elements.

FIG. 2 is a front view showing the collapsed configuration for transport and the extendable telescoping horizontal arm connecting dual vertical columns, with the extendable conveyor belt and detector housing.

FIG. 3 is a lateral view illustrating the telescoping arm that allows independent vertical adjustment of the columns to accommodate aligning with tilted bed positions.

FIG. 4 is a detail view illustrating the detector housing with planar displacement mechanism showing X-Y fine adjustment capabilities.

FIG. 5 is an illustration of the user interface concept showing controls for raising, lowering, tilting, and detector placement and removal, with locking mechanisms.

FIGS. 6A through 6D are sequential views illustrating the detector deployment process, wherein FIG. 6A shows placement adjacent to bed, FIG. 6B shows the conveyor component raised to horizontal alignment, FIG. 6C shows the conveyor aligned and deploying under patient, and FIG. 6D shows extended deployment.

FIG. 7 is a top plan view of detector housing assembly and illustrates the continuous repositioning capability of X-ray detector during an interventional radiology procedure.

FIG. 8 is a composite view showing positioning functionality during an interventional procedure with sequential X-ray frames tracking detector motion.

FIG. 9 is a flowchart illustrating the system initialization and positioning sequence.

FIG. 10 is a flowchart illustrating the detector deployment sequence with position monitoring.

FIG. 11 is a flowchart illustrating the detector retraction sequence.

FIG. 12 is a block diagram of the controller system architecture and electronics.

### DETAILED DESCRIPTION

The following detailed description refers to the accompanying drawings that show, by way of illustration, specific embodiments in which the invention may be practiced. These embodiments are described in sufficient detail to enable those skilled in the art to practice the invention. Other embodiments may be utilized, and structural, logical, and electrical changes may be made without departing from the scope of the invention. The various embodiments are not necessarily mutually exclusive, as some embodiments can be combined with one or more other embodiments to form new embodiments.

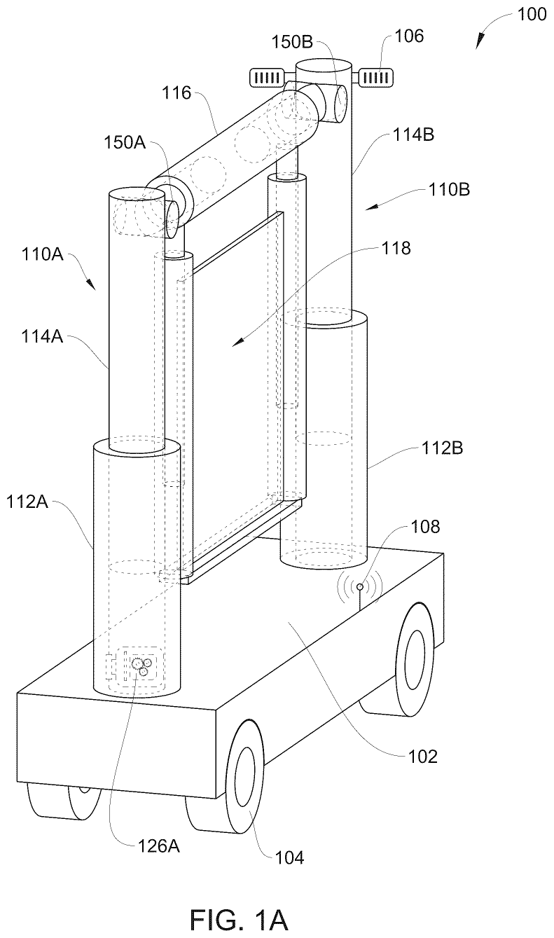

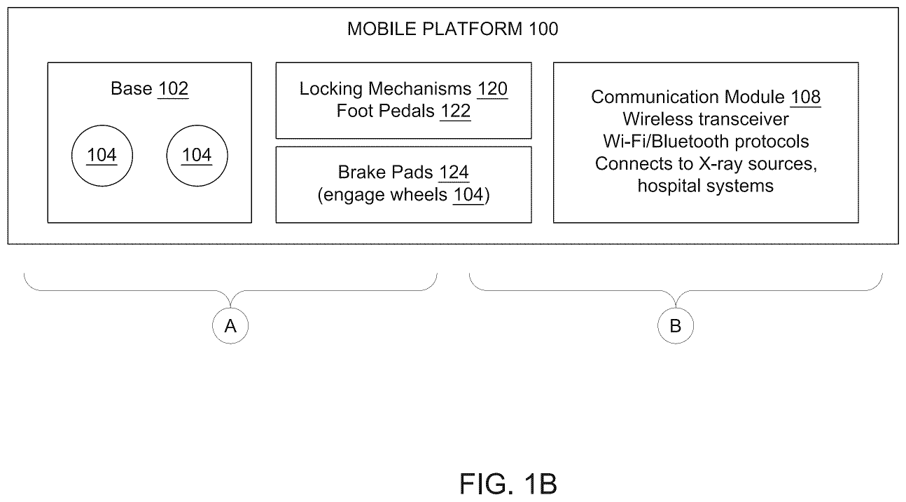

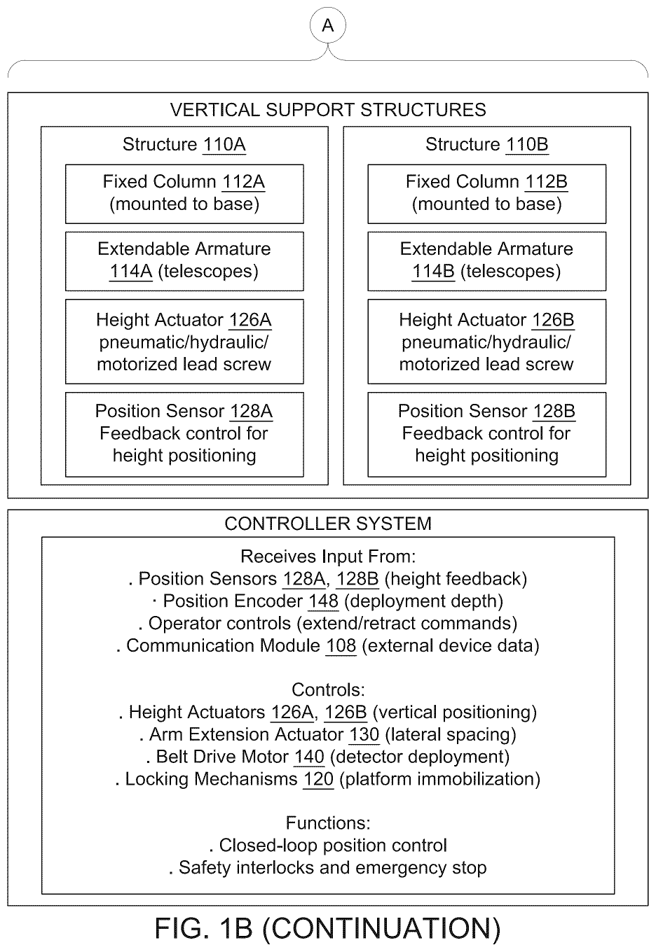

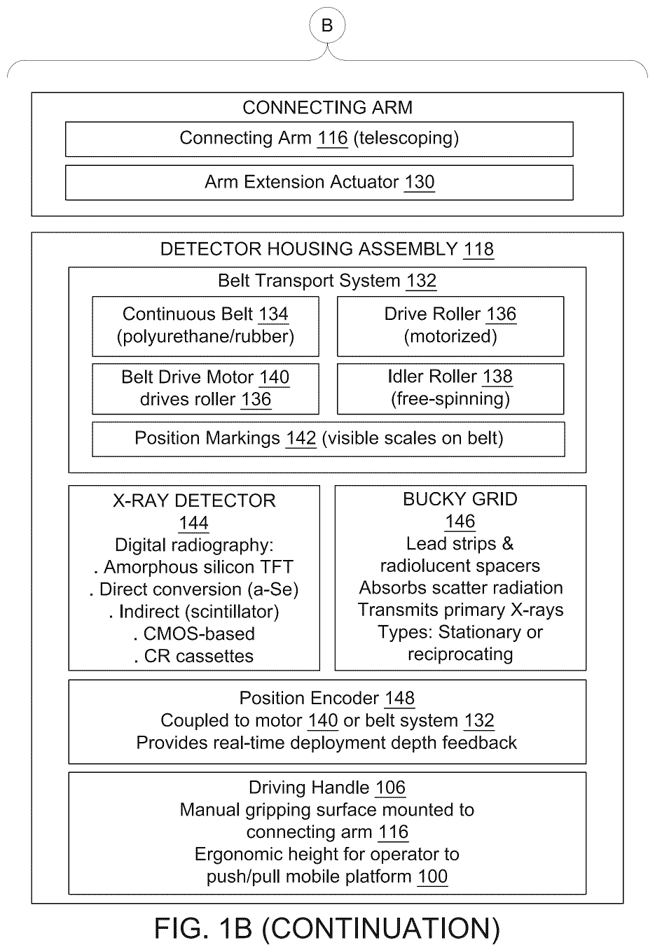

Referring to FIG. 1A, the bedside X-ray imaging system comprises bedside X-ray imaging system 100, dual vertical support structures 110A and 110B, and detector housing assembly 118. Bedside X-ray imaging system 100 forms the foundation of the system and provides mobility through hospital environments while maintaining stability during imaging operations. Bedside X-ray imaging system 100 comprises base 102 with multiple wheels 104 positioned at corners of base 102, enabling omnidirectional movement of the system. In the illustrated embodiment, four wheels 104 are provided, though alternative embodiments may employ six or more wheels for enhanced stability. Each wheel 104 is a swivel caster allowing rotation about a vertical axis, facilitating maneuvering through doorways and around obstacles in clinical settings. Wheels 104 comprise polyurethane wheel material providing floor protection, noise reduction, and adequate load capacity for the system mass plus detector and associated electronics. Typical wheel diameter ranges from three to six inches, with larger diameter wheels providing improved capability to traverse door thresholds and floor transitions.

Base 102 comprises a rigid structural assembly constructed from materials selected for strength, durability, and weight considerations. Suitable materials include aluminum alloys such as 6061-T6 aluminum providing high strength-to-weight ratio, steel tubing providing maximum rigidity, or composite materials including carbon fiber reinforced polymers providing lightweight construction with excellent mechanical properties. Base 102 defines a footprint dimensioned to provide stability during operation while maintaining maneuverability through standard hospital doorways typically measuring thirty-six inches in width. In various embodiments, base 102 measures approximately twenty-four to thirty-six inches in width and eighteen to thirty inches in depth, providing adequate base dimensions to resist tipping moments generated during belt assembly extension.

In some embodiments, the detector is deployed from a bedside platform by a conveyor belt mechanism that serves as the primary longitudinal transport. The belt can have a width of about 280 mm to about 360 mm with a thickness of about 1.5 mm to about 3.0 mm and a surface coefficient of friction of about 0.15 to about 0.35 against a radiolucent top skin. A leading nose portion may include a roller of about 12 mm to about 20 mm diameter with an edge radius of at least about 3 mm to improve patient comfort and reduce snag risk. Belt tensioning and tracking can be provided by an eccentric idler or screw take-up with about ±6 mm lateral adjustment and visible tracking marks for service verification. Typical longitudinal stroke is about 450 mm to about 650 mm with a commanded speed of about 40 mm/s to about 120 mm/s; stall is detected when measured torque or current exceeds about 120 percent of nominal for at least about 150 ms, at which point the controller halts motion and commands an automatic controlled retract to a safe position.

In other embodiments, the longitudinal deployment may be realized by alternative mechanisms that are functionally interchangeable with the belt transport. Examples include a roller-bed in which the detector housing rides on low-profile rollers along a guided channel with a driven center roller; a track-and-carriage sled translating on linear rails with a timing-belt or screw drive; a rack-and-pinion cantilever arm with captive carriage and positive detents; or a linear actuator driven slide with a guided shoe. Each alternative may include a serviceable cassette or module, a defined removal path, and tool callouts to enable replacement without disturbing the sealed detector cavity.

Driving handle 106 is mounted at an upper portion of the system and provides a manual gripping surface for an operator to push or pull bedside X-ray imaging system 100 during positioning and transport. Driving handle 106 extends horizontally and is positioned at an ergonomic height for operator access. In the illustrated embodiment, driving handle 106 is mounted to connecting arm 116 between the two vertical support structures, allowing the operator to apply motive force to the system while maintaining balance and control during maneuvering.

In some embodiments, the vertical support provides a detector datum height adjustment from about 480 mm to about 1,000 mm above the floor and accommodates patient-support tilt of about ±15 degrees while maintaining detector parallelism to the patient surface. The lift mechanism may be realized by dual motorized lead screws (for example about 10×3 mm pitch), a scissor lift sized for at least about 1.5 times the maximum working load, or a powered articulated arm with an anti-backdrive brake. Under a vertical load of about 300 N at mid-span, static deflection at the detector plane can be limited to about 3 mm or less, with lift acoustic emission not exceeding about 55 dBA at 1 m in typical operation.

Communication module 108 is mounted to the system structure and provides wireless connectivity capability. Communication module 108 comprises a wireless transceiver configured to establish communication with external devices such as mobile X-ray sources, hospital information systems, or remote-control interfaces. Communication module 108 implements Wi-Fi communication protocols, Bluetooth communication protocols, or both, enabling data exchange including position information, timing synchronization signals, image data, and system status information. Communication module 108 includes antenna elements for transmitting and receiving radio frequency signals.

First vertical support structure 110A and second vertical support structure 110B extend upward from bedside X-ray imaging system 100 in spaced-apart parallel relationship. First vertical support structure 110A comprises fixed column 112A mounted to base 102 and extendable armature 114A telescopically coupled to fixed column 112A. Second vertical support structure 110B comprises fixed column 112B mounted to base 102 and extendable armature 114B telescopically coupled to fixed column 112B. Fixed columns 112A and 112B provide rigid vertical support, while extendable armatures 114A and 114B translate vertically relative to their respective fixed columns to provide height adjustment capability.

In some embodiments, the platform collapses for transport to an envelope of not more than about 600 mm in width, about 800 mm in length, and about 1,300 mm in height while preserving a deployed width of about 700 mm to about 1,200 mm for bedside operation. The platform can include casters of about 125 mm to about 150 mm diameter with tread hardness of about 70 Shore A to about 90 Shore A and total-lock brakes; an individual caster load rating of at least about 150 kg; and a turning radius of not more than about 850 mm to facilitate maneuvering through hospital corridors and door thresholds of about 20 mm to about 25 mm height.

Extendable armature 114A slides vertically within or along fixed column 112A, and extendable armature 114B slides vertically within or along fixed column 112B. The telescoping construction enables independent or coordinated vertical translation of the armatures to align detector housing assembly 118 with patient support surfaces of various elevations and inclinations. The dual vertical support structure configuration provides enhanced stability and allows for tilt adjustment to accommodate non-horizontal patient support surfaces.

In some embodiments, the detector housing supports multiple detector formats, such as 24×30 cm and 35×43 cm plates, by use of calibrated shims or adapters with a dimensional tolerance of about ±0.5 mm and automatic detector recognition via a contact or near-field interface. The patient-facing top skin can be a radiolucent laminate, such as a carbon fiber epoxy layer of about 0.8 mm to about 1.2 mm thickness or an ultra-high-molecular-weight polyethylene film of about 0.5 mm to about 0.8 mm thickness, with a surface roughness of about Ra 0.8 μm to about 1.6 μm to facilitate low-shear sliding. The housing can provide a static load rating of about 1.6 kN over a 50 mm by 50 mm patch, include fluid-ingress control at about IPX4 on the patient side with defined drain paths, and incorporate cable or flex routing with a minimum bend radius of about 50 times the cable thickness and inrush-limited, hot-plug-tolerant power entry.

Connecting arm 116 extends horizontally between extendable armature 114A and extendable armature 114B, joining the two vertical support structures at their upper portions. Connecting arm 116 comprises telescoping construction enabling lateral adjustment of the spacing between first vertical support structure 110A and second vertical support structure 110B. The telescoping capability of connecting arm 116 allows the system to accommodate different bed widths and provides a collapsed configuration with reduced overall width for transport through doorways and corridors.

In some embodiments, after longitudinal deployment the detector is repositioned within the housing by a planar mechanism that provides about ±75 mm to about ±100 mm travel along each orthogonal axis with repeatability of about 0.5 mm or better. Suitable drives include lead screw stages (for example about 6×1 mm), toothed belt stages, compact linear motors, or rack-and-pinion drives, each operated under closed-loop control at about 200 Hz to about 500 Hz with jerk-limited motion profiles. Hard and soft limits bound the travel, and mechanical bumpers provide passive overtravel protection.

Detector housing assembly 118 is mounted between extendable armatures 114A and 114B via connecting arm 116 and comprises a belt-driven conveyor mechanism configured to translate longitudinally to deploy an X-ray detector and Bucky assembly beneath a patient. The Bucky assembly integrates within the conveyor belt structure, with the X-ray detector and Bucky grid housed in a sealed enclosure that forms an integral component of the belt transport system. The belt structure provides both the motive force for longitudinal translation and the structural housing for the detector and grid components. Detector housing assembly 118 extends horizontally from the region between the two vertical support structures along a deployment axis oriented perpendicular to the plane defined by vertical support structures 110A and 110B. The belt-driven mechanism translates the integrated detector-Bucky assembly from a retracted position adjacent to the vertical support structures to an extended position beneath a patient positioned on a patient bed.

Referring collectively to FIGS. 1A and 1B, a block diagram illustrates the internal components and subsystems of the bedside X-ray imaging system not visible in the external view of FIG. 1A. Bedside X-ray imaging system 100 incorporates locking mechanism 120 configured to immobilize the platform during imaging operations. Locking mechanism 120 prevent unwanted movement that could disrupt detector positioning or create safety hazards. In a first embodiment, locking mechanism 120 comprise individual wheel brakes associated with each wheel 104. Each wheel brake includes foot pedal 122 operable by an operator to engage brake pad 124 against the respective wheel 104. Foot pedals 122 are positioned on opposite sides of base 102 for accessibility from either side of a patient bed. Depression of foot pedal 122 causes brake pad 124 to press against wheel 104, preventing both rotation and swiveling of that wheel. In alternative embodiments, locking mechanism 120 may comprise central locking systems wherein a single actuator simultaneously engages brakes on multiple wheels, or may comprise deployable stabilizing feet that lower from base 102 to contact the floor and provide additional support points.

In some embodiments, safety is provided by a combination of proximity and load sensing with defined trip thresholds and interlocks. The leading edge may include force-sensing elements that trip at about 10 N to about 20 N, time-filtered to about 20 ms to about 50 ms; time-of-flight optical ranging can cover from about 20 mm to about 1,000 mm; and ultrasonic ranging can cover from about 200 mm to about 4,000 mm, with a fusion algorithm that halts motion on any hazard vote. An emergency stop channel can meet a performance level in line with ISO 13849 concepts, and the controller can enforce immobilizer engagement prior to any deployment or retraction command.

Height adjustment actuators 126A and 126B control the telescoping motion of extendable armatures 114A and 114B respectively. Height adjustment actuator 126A actuates the vertical translation of extendable armature 114A relative to fixed column 112A, and height adjustment actuator 126B actuates the vertical translation of extendable armature 114B relative to fixed column 112B. Height adjustment actuators 126A and 126B each comprise, in various embodiments, a pneumatic cylinder, hydraulic cylinder, motorized lead screw, or motorized rack and pinion mechanism. Pneumatic cylinder embodiments utilize compressed air supplied from an onboard air compressor or external air line to drive piston motion, providing smooth and quiet height adjustment. Hydraulic cylinder embodiments utilize pressurized hydraulic fluid to generate higher forces for lifting heavier loads. Motorized lead screw embodiments comprise an electric motor rotating a threaded shaft engaged with a threaded nut fixed to the respective extendable armature, providing precise position control and holding capability without continuous power consumption.

Position sensors 128A and 128B provide feedback regarding the current extended height of extendable armatures 114A and 114B respectively, enabling closed-loop control by a system controller. Position sensors 128A and 128B may comprise linear potentiometers, linear variable differential transformers, magnetic position sensors, or optical encoders coupled to height adjustment actuators 126A and 126B.

In some embodiments, the system establishes a calibrated relationship among a chassis frame {B}, a conveyor frame {C}, a detector frame {D}, a world or room frame {W}, and a source frame {S}. Calibration can be achieved by one or more of: fiducial-based vision using coded planar targets, optical beacon triangulation, or radio-frequency ranging. The controller can compute transforms T_{BC}, T_{CD}, and T_{SW} and derive T_{SD} with a translational error not exceeding about 1.0 mm root-mean-square at the detector plane and a rotational error not exceeding about 0.5 degrees, with time synchronization provided by a precision time protocol and automatic recalibration upon drift detection.

Arm extension actuator 130 controls the telescoping extension and retraction of connecting arm 116, adjusting the lateral spacing between first vertical support structure 110A and second vertical support structure 110B. This adjustability enables the system to accommodate different bed widths and provides a collapsed configuration for transport through doorways and corridors.

In some embodiments, communication interfaces can include isolated wired Ethernet (for example 1000BASE-T) and wireless links implementing IEEE 802.11ax and Bluetooth Low Energy. The controller can exchange modality worklist and image objects via DICOM services and patient context via HL7 or FHIR while maintaining role-based access control, signed over-the-air updates with rollback, and audit logging suitable for clinical environments.

Detector housing assembly 118 comprises belt transport system 132 configured to translate longitudinally relative to the vertical support structures. Belt transport system 132 includes continuous belt 134 trained around drive roller 136 and idler roller 138. Drive roller 136 is coupled to belt drive motor 140, which rotates drive roller 136 to advance belt 134 in forward or reverse directions. Belt 134 comprises a flexible material such as polyurethane-coated fabric, reinforced rubber, or woven synthetic fiber providing adequate tensile strength. The belt transport system slides the detector housing into position beneath a patient who remains stationary on the patient support surface throughout deployment, with the belt and integrated housing passing between the mattress and bed frame without contacting or moving the patient. Integrated within belt 134 is a rigid detector-Bucky housing structure that moves with the belt during deployment and retraction. This integrated housing protects X-ray detector 144 and Bucky grid 146 while providing a low-profile form factor that slides beneath patients and mattresses. The housing structure integrated into belt 134 includes radiolucent patient-facing surfaces, cable management for detector power and data connections, and structural reinforcement to maintain detector alignment during translation. In some embodiments, the belt can include multiple belts that each move in discrete directions.

Belt 134 includes position markings 142 visible along an exterior surface of belt 134. Position markings 142 provide a visual indication to an operator of the deployment depth of detector housing assembly 118 during belt extension. Position markings 142 may comprise graduated scales printed, embossed, or otherwise applied to belt 134, indicating distance in units such as inches or centimeters from a reference starting position. As belt 134 advances, different position markings 142 become visible adjacent to a reference indicator on detector housing assembly 118, allowing an operator to monitor how far the detector and Bucky assembly have been deployed beneath a patient.

In some embodiments, visual guidance can be provided by projecting or displaying a field-of-view overlay that is registered to the detector frame {D}. In some embodiments, a camera operating at about 1080p and about 60 frames per second with end-to-end overlay latency of not more than about 80 ms can support alignment feedback. This feature is particularly useful in remote device use.

Mosaicking can employ feature-based registration with gain compensation and blending to yield composite images with a geometric error of about 1.5 mm or less across a combined field exceeding a single detector dimension, using stored detector pose metadata from the planar mechanism. This allows for the creation of a large X-Ray composite.

X-ray detector 144 is integrated within the belt structure of detector housing assembly 118 and comprises a digital radiography detector. The detector is installed within a sealed enclosure that forms part of the conveyor belt assembly during factory configuration or field service procedures. This service-based installation model differs from systems requiring daily insertion and removal of detectors by operators, instead providing a sealed and protected detector that deploys as an integral unit with the belt transport system. X-ray detector 144 may utilize amorphous silicon thin-film transistor arrays, direct conversion detectors utilizing amorphous selenium, indirect conversion detectors utilizing scintillator materials coupled to photodiodes, CMOS-based detectors, or computed radiography cassettes. X-ray detector 144 is oriented with its active detection surface facing upward toward the patient-facing surface of the belt-integrated housing to receive X-rays that pass through the patient.

For interventional procedure applications, a smaller CMOS-based detector may be employed within the same deployment envelope, offering high frame rates of 30 fps or greater to enable active tracking of catheter or other interventional tool displacement in real time. While CMOS detectors have greater thickness than conventional flat-panel detectors, the reduced active area and high temporal resolution make them suitable for dynamic interventional imaging within the same detector housing assembly.

Bucky grid 146 is integrated within the belt structure of detector housing assembly 118, positioned between the patient-facing surface and X-ray detector 144. Bucky grid 146 comprises alternating lead strips and radiolucent spacers configured to absorb scatter radiation while transmitting primary X-rays to X-ray detector 144. The lead strips are oriented to allow primary X-rays traveling perpendicular to the detector surface to pass through while blocking scattered X-rays traveling at oblique angles. The Bucky grid is permanently installed within the belt-integrated housing structure, maintaining fixed spatial relationship to X-ray detector 144 during all deployment and retraction operations. In certain embodiments, Bucky grid 146 is stationary relative to the detector, while in other embodiments, Bucky grid 146 incorporates a reciprocating mechanism integrated into the belt housing that oscillates the grid during X-ray exposure to eliminate grid line artifacts from the resulting image.

Position encoder 148 is operatively coupled to belt drive motor 140 or to belt transport system 132 and provides position feedback signals indicating the current extension distance of detector housing assembly 118 from the retracted position. Position encoder 148 may comprise a rotary encoder measuring rotations of drive roller 136, a linear encoder measuring belt displacement, or other position sensing means providing accurate real-time deployment depth information to the system controller.

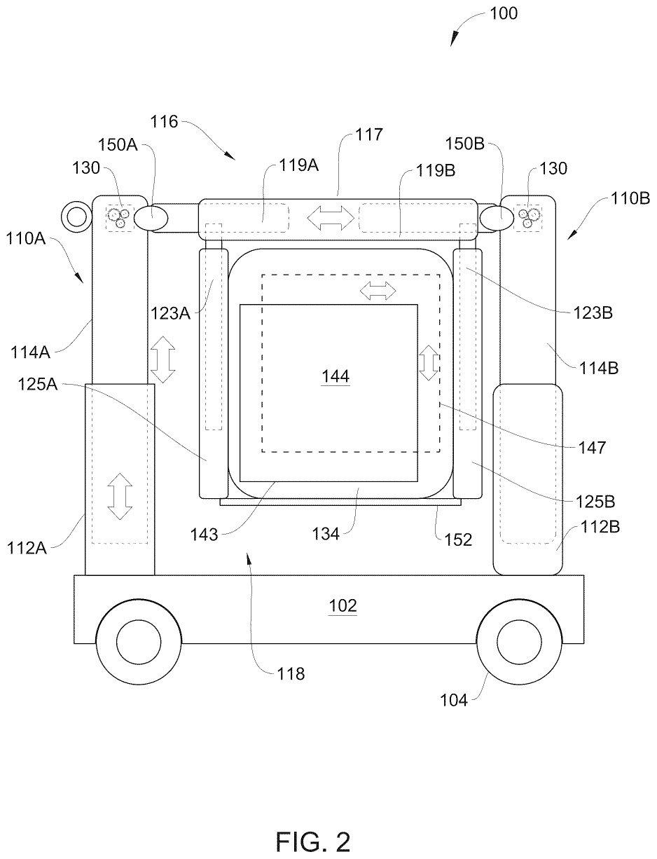

Referring to FIG. 2, a front view of bedside X-ray imaging system 100 illustrates a dual vertical support structure configuration, a connecting arm adjustment mechanism, and detector housing assembly deployment capability. The front view shows bedside X-ray imaging system 100 comprising base 102 with wheels 104 positioned at opposite lateral edges.

First vertical support structure 110A is positioned on the left side of base 102, and second vertical support structure 110B is positioned on the right side of base 102. First vertical support structure 110A and second vertical support structure 110B extend upward from base 102. First vertical support structure 110A comprises fixed column 112A mounted to base 102. Second vertical support structure 110B comprises fixed column 112B mounted to base 102.

First extendable armature 114A and second extendable armature 114B provide vertical telescoping for height alignment with the patient support surface and are distinct from the belt conveyor mechanism used for longitudinal deployment of the detector housing assembly 118.

Connecting arm 116 extends horizontally between an upper portion of first extendable armature 114A and an upper portion of second extendable armature 114B. Connecting arm 116 comprises central strut 117, first telescoping armature 119A, and second telescoping armature 119B. First telescoping armature 119A couples to first extendable armature 114A. Second telescoping armature 119B couples to second extendable armature 114B. Central strut 117 translates along first telescoping armature 119A and second telescoping armature 119B, enabling lateral adjustment of spacing between first vertical support structure 110A and second vertical support structure 110B. This telescoping capability allows bedside X-ray imaging system 100 to accommodate different bed widths and provides a collapsed configuration for transport.

One actuator 130 is positioned on first telescoping armature 119A. Another actuator 130 is positioned on second telescoping armature 119B. The actuators 130 each comprise servos or motors that drive translation of central strut 117 along first telescoping armature 119A and second telescoping armature 119B.

First joint 150A connects connecting arm 116 to first extendable armature 114A. Second joint 150B connects connecting arm 116 to second extendable armature 114B. First joint 150A and second joint 150B provide rotational articulation enabling rotation of central strut 117 relative to first extendable armature 114A and second extendable armature 114B. In various embodiments, first joint 150A and second joint 150B comprise pivot joints, ball joints, or universal joints enabling one or more degrees of rotational freedom.

In the configuration shown in FIG. 2, an X-axis planar guideway laterally repositions detector housing assembly 118. A first fixed shaft 123A is supported proximate the interface of central strut 117 and the left side of the assembly, and a second fixed shaft 123B is supported proximate the interface of central strut 117 and the right side. A first linear guide track 125A is coupled to central strut 117 and slides along shaft 123A; a second linear guide track 125B is coupled to central strut 117 and slides along shaft 123B. Together, shafts 123A and 123B with guide tracks 125A and 125B form the lateral guideway for detector housing assembly 118.

Detector housing assembly 118 is attached to central strut 117. Detector housing assembly 118, first fixed shaft 123A, second fixed shaft 123B, and central strut 117 form a rigid assembly. First joint 150A and second joint 150B enable rotation of this rigid assembly relative to first extendable armature 114A and second extendable armature 114B. This rotation pivots detector housing assembly 118 from a vertical orientation to a horizontal orientation or any position therebetween. The vertical orientation is used for transport or storage. The horizontal orientation is used for deployment beneath a patient.

Detector housing assembly 118 comprises a substantially rectangular enclosure housing X-ray detector 144 and conveyor belt mechanism 134 integrated within the enclosure. Conveyor belt mechanism 134 provides the primary longitudinal deployment mechanism for inserting X-ray detector 144 beneath a patient. Within detector housing assembly 118, a solid rectangular outline 143 represents X-ray detector 144 in a first position, and a dotted rectangular outline 147 represents the detector in a second position. The two outlines 143 and 147 depict first and second positions of the same X-ray detector 144, illustrating planar displacement capability within detector housing assembly 118. This fine motor control enables repositioning of the X-ray detector after initial conveyor deployment, providing precise alignment with an X-ray source or real-time tracking of interventional devices during procedures without requiring retraction and reinsertion of the entire conveyor assembly. A horizontal double-headed arrow on detector housing assembly 118 indicates a deployment direction along which detector housing assembly 118 translates to extend from a retracted position shown in the FIG. to an extended position beneath a patient.

Bed sensor 152 is mounted at a leading edge of detector housing assembly 118. Bed sensor 152 is positioned to detect proximity to or contact with a patient bed during deployment. Bed sensor 152 comprises a proximity sensor, a contact switch, or an optical sensor configured to generate a signal when detector housing assembly 118 approaches or contacts bed structures. Detection of bed contact by bed sensor 152 provides feedback to a controller system or an operator, indicating that further extension should be halted to prevent collision or excessive pressure against the patient bed. In automated deployment embodiments, bed sensor 152 triggers an automatic stop of a belt drive motor, preventing over-extension. In manually controlled embodiments, bed sensor 152 activates a warning indicator alerting the operator to cease extension.

The front view illustrates three primary degrees of freedom provided by bedside X-ray imaging system 100: vertical translation of first extendable armature 114A and second extendable armature 114B relative to fixed column 112A and fixed column 112B respectively, indicated by vertical double-headed arrows on first vertical support structure 110A and second vertical support structure 110B; lateral translation via first linear guide track 125A sliding over first fixed shaft 123A and second linear guide track 125B sliding over second fixed shaft 123B in combination with translation of central strut 117 along first telescoping armature 119A and second telescoping armature 119B, indicated by horizontal double-headed arrows on connecting arm 116; and longitudinal translation of detector housing assembly 118 via conveyor belt mechanism 134, indicated by the horizontal double-headed arrow on detector housing assembly 118. These combined degrees of freedom enable bedside X-ray imaging system 100 to position the X-ray detector beneath patients on beds of varying configurations, heights, and widths without requiring integration into bed structure.

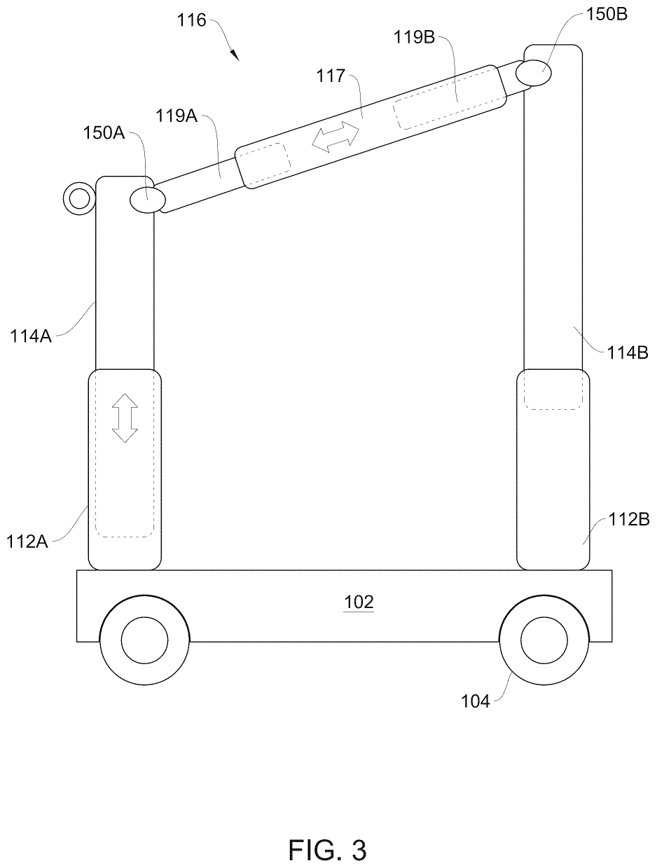

Referring to FIG. 3, a lateral side view of the bedside X-ray imaging system illustrates the independent height adjustment capability of the dual vertical support structures and the resulting angular orientation of connecting arm 116 when the vertical support structures are adjusted to different heights.

The lateral view shows bedside X-ray imaging system 100 comprising base 102 with wheels 104. First vertical support structure 110A is visible in the foreground, and second vertical support structure 110B is visible in the background. Each vertical support structure comprises fixed column 112A or 112B extending upward from base 102, with extendable armature 114A or 114B telescopically engaged with the respective fixed column. The vertical double-headed arrow on first vertical support structure 110A indicates the telescoping motion range, wherein extendable armature 114A translates vertically relative to fixed column 112A.

In the configuration illustrated in FIG. 3, first vertical support structure 110A is extended to a first height, while second vertical support structure 110B is extended to a different second height, creating a height differential between the two vertical support structures. Connecting arm 116 extends between extendable armature 114A and extendable armature 114B at the upper portions of the vertical support structures. Due to the height differential between the two vertical support structures, connecting arm 116 is oriented at an angle relative to horizontal. The horizontal double-headed arrow on connecting arm 116 indicates the telescoping capability that enables lateral spacing adjustment between the two vertical support structures.

Joints 150A and 150B connect connecting arm 116 to extendable armatures 114A and 114B respectively at the angled interface locations. The rotational articulation provided by joints 150A and 150B accommodates the angular orientation of connecting arm 116, allowing the connecting arm to maintain structural integrity while spanning between vertical support structures positioned at different heights.

The independent height adjustment capability illustrated in FIG. 3 enables the system to align the detector housing assembly parallel to patient support surfaces that are not horizontal. Hospital beds frequently position patients with the head section elevated for respiratory support or patient comfort, creating an inclined patient support surface. By independently adjusting first vertical support structure 110A to a different height than second vertical support structure 110B, the system establishes an angled orientation of connecting arm 116 that matches the inclination angle of the tilted patient support surface. The detector housing assembly, when mounted to connecting arm 116, thereby maintains parallel alignment with the tilted patient support surface throughout the deployment path, ensuring proper positioning of the X-ray detector beneath the patient without interference with bed structures.

The lateral view of FIG. 3 further illustrates the overall height range of the vertical support structures. Fixed columns 112A and 112B provide rigid structural support over the full height range, while extendable armatures 114A and 114B translate within or along the fixed columns to achieve the desired vertical positioning. The telescoping construction enables the system to accommodate patient beds with support surface heights ranging from approximately twenty-four inches to forty-eight inches above the floor, covering the typical range of hospital bed configurations including pediatric beds, standard adult beds, and intensive care unit beds.

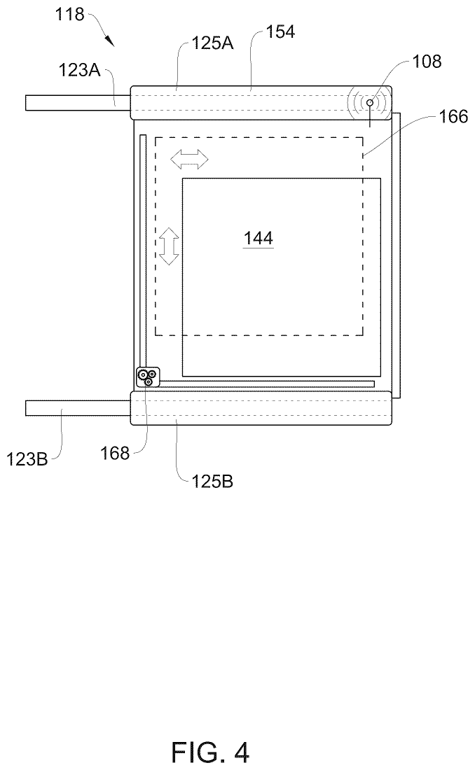

Referring to FIG. 4, a top plan view of detector housing assembly 118 illustrates the planar displacement mechanism and internal positioning components that enable fine two-dimensional adjustment of X-ray detector 144 within the detector housing envelope. Detector housing assembly 118 is shown from above, with the top surface removed to reveal the internal components. The outer perimeter represents housing enclosure 154. X-ray detector 144 is shown as the solid gray rectangle positioned within housing enclosure 154.

Dashed outline 166 surrounding X-ray detector 144 represents the range of motion envelope within which X-ray detector 144 can be positioned by planar displacement mechanism 168. The horizontal double-headed arrow indicates translation capability along the X-axis, and the vertical double-headed arrow indicates translation capability along the Y-axis. These orthogonal motion axes enable X-ray detector 144 to be positioned at any location within the two-dimensional area defined by dashed outline 166.

A first fixed shaft 123A is disposed along an inner left-hand margin of housing enclosure 154 and cooperates with a first linear guide track 125A that slides on the shaft 123A to provide translation of X-ray detector 144 along a first axis within motion envelope 166. A second fixed shaft 123B is arranged orthogonally to the first and extends along a lower margin of the enclosure. A second linear guide track 125B slides on shaft 123B and is mechanically coupled with the first guide track so that combined motion of guide tracks 125A, 125B over fixed shafts 123A, 123B effects planar two-degree-of-freedom positioning of X-ray detector 144 inside detector housing assembly 118. In the illustrated embodiment, planar displacement mechanism 168 drives at least one of the guide tracks 125A, 125B, with the orthogonal degree of freedom provided by either a crosshead linkage or a separate drive, and limit features are provided at the ends of shafts 123A, 123B.

Planar displacement mechanism 168 is shown in the lower left corner of detector housing assembly 118. Planar displacement mechanism 168 comprises actuator assemblies configured to translate X-ray detector 144 along orthogonal X and Y axes. In various embodiments, planar displacement mechanism 168 may comprise motorized lead screw assemblies, belt and pulley systems, rack and pinion mechanisms, linear motor stages, piezoelectric actuators, or electromagnetic positioning stages.

Communication module 108 provides wireless connectivity via Wi-Fi and Bluetooth protocols, enabling detector housing assembly 118 to exchange data with external X-ray sources, including position information for coordinate system calibration, synchronization signals for timed image acquisition, and image data transfer to remote displays or hospital information systems.

The planar displacement capability illustrated in FIG. 4 enables two primary operational modes. In a first mode, after the conveyor belt system deploys detector housing assembly 118 to a position beneath a patient, an operator commands planar displacement mechanism 168 to make fine positioning adjustments of X-ray detector 144 to optimize alignment with the anatomical region of interest or with the X-ray source position, without requiring retraction and redeployment of the entire detector housing assembly.

The planar displacement mechanism operates independently from and subsequent to the belt transport system. The belt transport system provides the primary longitudinal deployment function, extending the detector housing assembly from the retracted position adjacent to the vertical support structures to an extended position beneath the patient. Once the belt transport system positions the housing at the target location and stops, the housing remains stationary beneath the patient for the duration of the imaging procedure. The planar displacement mechanism then provides secondary fine positioning by translating the X-ray detector within the stationary housing envelope. This two-stage positioning architecture separates coarse positioning accomplished by belt transport from fine positioning accomplished by planar displacement, enabling rapid detector deployment followed by precise alignment or continuous tracking without requiring retraction and redeployment of the entire housing assembly via the belt transport system.

In a second mode applicable to interventional radiology procedures, planar displacement mechanism 168 continuously repositions X-ray detector 144 during the procedure to track an interventional device such as a catheter or guidewire as it advances through patient anatomy. As the interventional device approaches the edge of the detector field of view, planar displacement mechanism 168 automatically translates X-ray detector 144 in the appropriate direction to maintain the device within the field of view. Multiple X-ray images acquired at different detector positions can be stored with associated position metadata, enabling post-procedure image mosaicking to generate composite images showing extended fields of view exceeding the dimensions of X-ray detector 144.

User interface control panel 178 is labeled “XDR3” and comprises a handheld or console-mounted control interface with multiple control buttons arranged in a logical functional layout. The control panel provides tactile button controls for all primary system operations, enabling the operator to position the mobile platform, adjust the vertical support structures, deploy the detector housing assembly, and engage the locking mechanisms without requiring access to multiple separate control locations on the system.

The control buttons include a “raise” button for commanding upward vertical translation of the vertical support structures, a “lower” button for commanding downward vertical translation, and a “tilt” button for commanding differential height adjustment between the first and second vertical support structures to establish an angled orientation of the connecting arm. The tilt button enables the operator to match the inclination angle of tilted patient support surfaces.

A “place” button commands the belt drive motor to extend the detector housing assembly from the retracted position toward the extended position beneath a patient. The operator depresses and holds the place button to initiate and maintain belt advancement, or in alternative embodiments, a single press commands automated extension to a programmed target position. A “remove” button commands the belt drive motor to retract the detector housing assembly from the extended position back to the retracted position adjacent to the vertical support structures.

A “lock” button commands engagement of the locking mechanisms, activating wheel brakes or deploying stabilizing feet to immobilize the mobile platform. An “unlock” button commands release of the locking mechanisms, allowing free mobility of the mobile platform for repositioning.

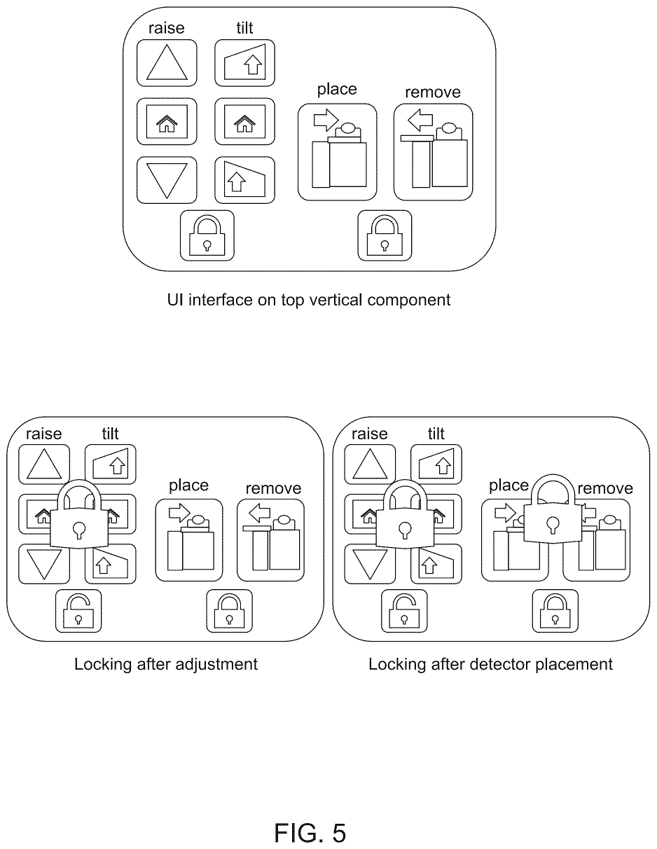

FIG. 5 illustrates operational states demonstrating the sequential workflow for system setup and detector deployment. User interface control panel 178 shows the initial state with all control functions accessible. User interface control panel 180, labeled “Locking after adjustment,” shows the interface state after the operator has adjusted the platform position and vertical support structure heights, with a lock indicator overlay showing that certain functions are disabled once the locking mechanisms are engaged to prevent platform movement. User interface control panel 182, labeled “Locking after detector placement,” shows the interface state after the detector housing assembly has been deployed, with additional functions locked to prevent inadvertent motion during imaging operations.

The lock indicator overlay, represented by a padlock icon covering multiple control buttons, visually indicates to the operator which functions are currently inhibited by safety interlocks. When the locking mechanisms are engaged, the raise, lower, and tilt functions are disabled to prevent height adjustment that could destabilize the locked platform. After detector deployment, additional interlocks prevent simultaneous adjustment operations that could interfere with imaging procedures or cause collision hazards.

The sequential locking behavior illustrated in FIG. 5 implements a safety protocol ensuring that system motions occur only in appropriate operational sequences. The operator must first position and lock the mobile platform before deploying the detector housing assembly, and must retract the detector before unlocking the platform for repositioning. This interlock logic prevents unsafe conditions such as detector deployment from an unlocked mobile platform or platform movement while the detector is extended beneath a patient.

In various embodiments, user interface control panel 178 comprises a physical handheld pendant control with tactile buttons, a touchscreen display presenting virtual control buttons, or a wireless remote-control device communicating with the controller system via the wireless communication module described in FIG. 1B. The control panel provides visual feedback through button illumination, display screen status messages, or color-coded indicators showing the current operational state and available functions.

The text to the right of the control panel illustrations in FIG. 5 provides a summary of key user interface interactions, stating: “Raising/lowering and tilt adjustment of the platform,” “Placement and removal of detector,” and “Locking mechanism (SW &/or physical) following detector deployment.” These descriptions correspond to the functional capabilities provided by the control buttons on user interface control panels 178, 180, and 182.

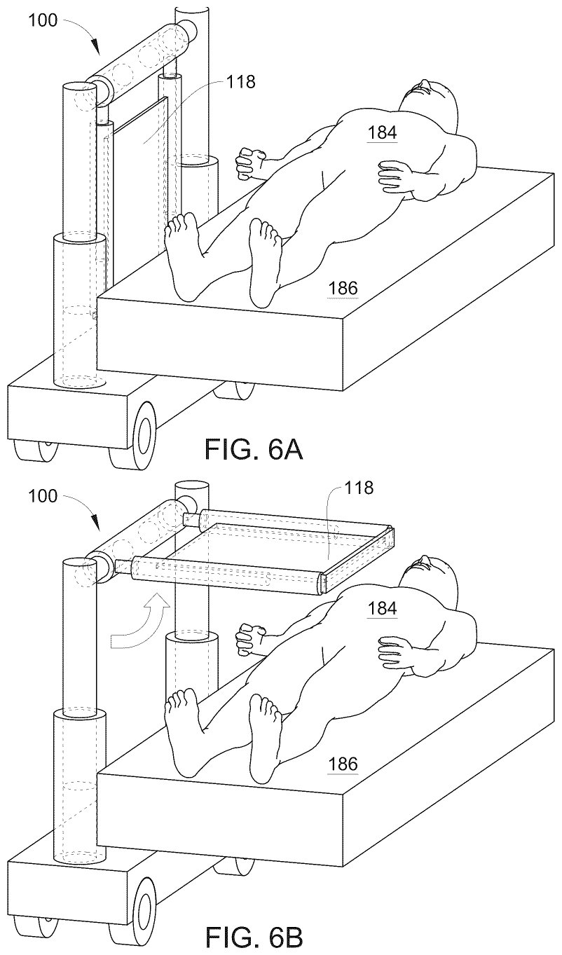

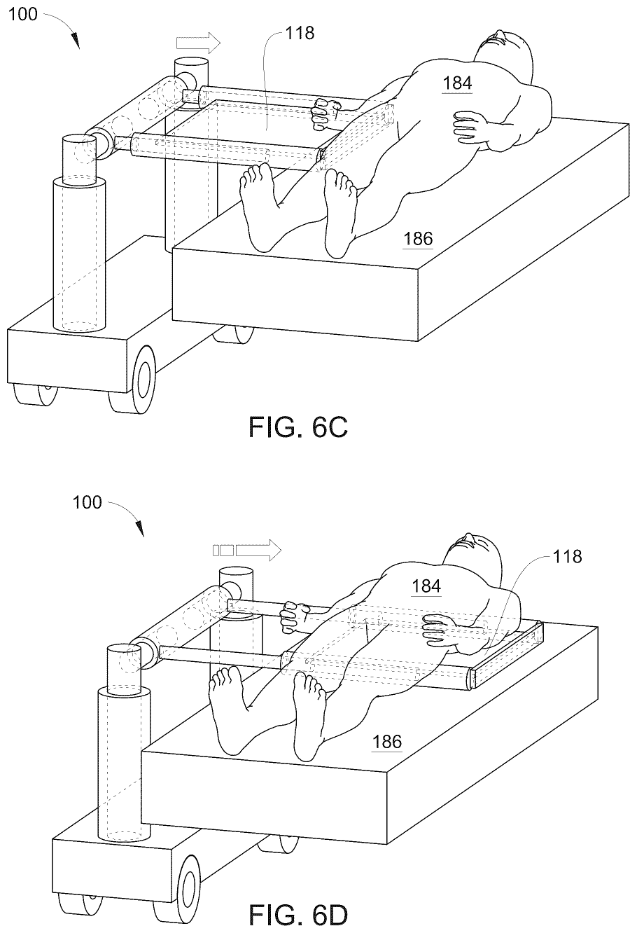

Referring to FIGS. 6A through 6D, a sequential operational deployment process illustrates the positioning and extension of bedside X-ray imaging system 100 to deploy detector housing assembly 118 beneath patient 184 positioned on patient bed 186. FIG. 6A, labeled “Placement by bed,” shows the initial positioning step. Bedside X-ray imaging system 100 is positioned on the floor adjacent to patient bed 186, which supports patient 184 lying on the patient support surface. At this stage, the system is in a compact transport configuration with detector housing assembly 118 in a vertical or retracted orientation close to the vertical support structures. The operator maneuvers bedside X-ray imaging system 100 until the system is properly aligned adjacent to patient bed 186, then engages the locking mechanisms to immobilize the platform.

FIG. 6B, labeled “Conveyor component raised to horizontal,” shows the height adjustment step. The vertical support structures have been extended to align detector housing assembly 118 with the height of the patient support surface. The curved arrow indicates the rotational or elevational motion of detector housing assembly 118 from the initial vertical position shown in FIG. 6A to the horizontal aligned position shown in FIG. 6B. The operator actuates height adjustment controls to raise detector housing assembly 118 until it is at the same elevation as the patient support surface, ensuring the deployment path will be parallel to the patient and will allow detector housing assembly 118 to slide beneath patient 184 without interference with bed structures.

FIG. 6C, labeled “Conveyor component aligned, sensing and deploying under patient,” shows the deployment initiation step. The belt transport system is actuated to begin extending detector housing assembly 118 from the retracted position toward patient 184. The horizontal arrow indicates the deployment direction. As detector housing assembly 118 extends, it passes over the edge of the bed frame and begins to slide beneath the mattress and patient 184. Proximity sensors monitor for contact with bed structures during deployment.

FIG. 6D, labeled “Extended conveyor deployment,” shows the fully deployed configuration. Detector housing assembly 118 has reached the extended position beneath patient 184, with the detector positioned under the anatomical region of interest. The dashed lines indicate the position of detector housing assembly 118 beneath patient 184 and the mattress. The extended arrow shows the full deployment range. At this point, the belt transport system stops, holding detector housing assembly 118 at the current position. The system is now ready for X-ray image acquisition. After image acquisition is complete, the operator actuates retraction controls to reverse the belt transport system and withdraw detector housing assembly 118 back to the retracted position.

The sequential views of FIGS. 6A through 6D demonstrate the complete workflow for positioning the system adjacent to a patient bed, adjusting the height to align with the patient support surface, deploying the detector beneath the patient without manual patient lifting, acquiring images, and retracting the detector. This workflow eliminates the need for multiple healthcare workers to lift and reposition patients, reducing physical strain on staff and improving patient comfort and safety during bedside radiographic imaging procedures.

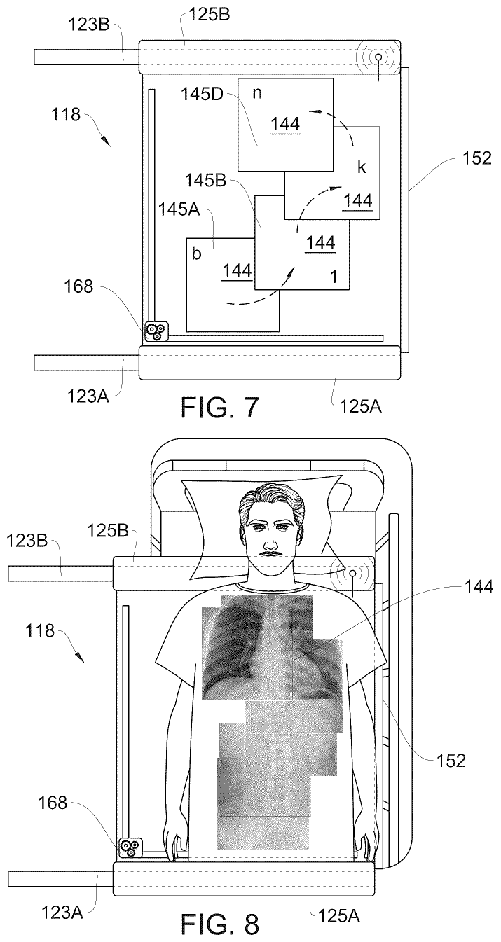

Referring to FIG. 7, a top plan view of detector housing assembly 118 illustrates continuous in-plane repositioning of X-ray detector 144 during an interventional procedure. The outer perimeter depicts housing enclosure 154. Within motion envelope 166, planar displacement mechanism 168 translates detector 144 in the X-Y plane parallel to the patient support surface.

Four detector positions are labeled 145A, 145B, 145C, and 145D. Position 145A represents an initial alignment at the start of the tracking sequence. Positions 145B and 145C depict intermediate translations executed by planar displacement mechanism 168 to maintain an interventional device within the detector field of view as the device advances through patient anatomy. Position 145D represents a terminal pose used to complete a stitched mosaic. Dashed arrows indicate representative translation paths between these poses. Detector 144 is common to all poses; only its planar position changes.

At each pose, the system acquires an image and stores pose metadata identifying the X-Y coordinates of detector 144 relative to the housing reference frame. The pose metadata enables real-time device tracking and post-procedure registration. A mosaicking process aligns images acquired at 145A through 145D based on the recorded poses and overlapping anatomical features to generate a composite image with an extended field of view.

The continuous repositioning illustrated in FIG. 7 increases the effective imaging range beyond the physical dimensions of detector 144 without retracting detector housing assembly 118 via belt transport system 132, supporting uninterrupted bedside workflow during time-sensitive interventional procedures.

Referring to FIG. 8, a composite illustration demonstrates the image mosaicking capability during an interventional radiology procedure, showing multiple X-ray images acquired at different detector positions combined to create an extended field of view covering a larger anatomical region than the physical dimensions of X-ray detector 144.

Patient 184 is positioned on patient bed 186, viewed from above. Detector housing assembly 118 is positioned beneath patient 184 with planar displacement mechanism 168 visible at the lower left corner, labeled “XY-fine motor in plane adjustment.” The rectangular outline represents the perimeter of detector housing assembly 118 beneath the patient.

Multiple overlapping rectangular frames within the detector housing outline represent individual X-ray images acquired at different positions of X-ray detector 144 as planar displacement mechanism 168 continuously repositioned the detector during an interventional procedure. Each rectangular frame corresponds to a single X-ray exposure captured at a specific X-Y detector position, with the frames overlapping at their edges where adjacent images share common anatomical features. In some instances, movement or displacement can be done in increments to prevent motion blur according to standard exposure practices.

In certain interventional embodiments, the detector housing assembly may incorporate a smaller CMOS-type X-ray detector dimensioned to occupy a reduced footprint within the housing envelope. The CMOS detector architecture provides frame rates exceeding 30 fps, enabling rapid sequential image acquisition to track catheter or guidewire displacement during device advancement. The high frame rate capability supports fluoroscopy-like visualization at the bedside without requiring transport to a dedicated interventional suite. The planar displacement mechanism repositions the smaller detector within the housing to maintain the interventional device within the active imaging area, while the reduced detector dimensions allow faster repositioning response.

The X-ray images within the frames show portions of patient anatomy including the thoracic region with visible lung fields, ribs, spine, and soft tissue structures. The largest central frame shows a chest X-ray image capturing the patient's thorax. Surrounding smaller frames show adjacent anatomical regions captured as planar displacement mechanism 168 translated X-ray detector 144 to track an interventional device advancing through the patient's vasculature or other anatomical pathways.

The overlapping arrangement of the image frames illustrates the image registration and mosaicking process performed by the controller system after image acquisition. The controller system utilizes position metadata indicating the X-Y coordinates of X-ray detector 144 at each image acquisition moment. Image processing algorithms identify overlapping anatomical features between adjacent images, apply geometric transformations to align the images in a common coordinate frame based on the known detector positions, and blend the overlapping regions to create a seamless composite image.

The resulting composite X-ray image spans an extended field of view significantly larger than the approximately fourteen-inch by seventeen-inch active area of a typical digital radiography detector. The system includes contrast tracking capabilities for applications such as angiography, venography, and arteriography. The controller monitors contrast agent flow through vascular structures in real-time and automatically commands planar displacement mechanism 168 to reposition X-ray detector 144 to maintain the contrast-enhanced vessels within the field of view. This dynamic tracking enables comprehensive visualization of vascular anatomy during contrast injection procedures without requiring manual detector repositioning or repeated contrast administration. The contrast tracking functionality integrates with the image mosaicking capability, generating composite angiographic images showing extended vascular pathways from injection site through distal vasculature.

The X-ray mosaic creation process serves multiple clinical and technical purposes. First, mosaicked images provide a global overview of interventional procedures, enabling clinicians to visualize the complete device trajectory and anatomical context beyond the constraints of a single detector field of view. Second, the mosaicking algorithm integrates information from individual image frames to increase overall image quality through multi-frame averaging and noise reduction. Third, the system accounts for both large anatomical motions such as respiratory excursion and fine motions such as cardiac pulsation, using position metadata and image registration to compensate for patient movement between frame acquisitions. Fourth, the system can integrate imaging information from the same procedure over time or across multiple patients for classes of procedures, building reference databases that support procedural planning, quality assessment, and training applications. Position metadata stored with each image frame includes X-Y detector coordinates, timestamp, exposure parameters, and synchronization data, enabling retrospective image processing and procedure analysis.

This extended field of view enables visualization of the complete trajectory of an interventional device such as a catheter from its insertion point through intermediate anatomical landmarks to its final positioned location, providing the interventional radiologist with comprehensive guidance for device placement without requiring multiple separate imaging sessions or patient repositioning.

Communication module 108 with wireless connectivity enables real-time transmission of the acquired images and position data to external display systems, allowing remote monitoring of the interventional procedure and collaborative decision-making among clinical team members at different locations within the hospital.

The image mosaicking capability illustrated in FIG. 8 represents a technical advancement over conventional bedside X-ray imaging, which is limited to the fixed field of view of a single detector position. By combining continuous detector repositioning via planar displacement mechanism 168 with automated image mosaicking, the system extends the effective imaging range while maintaining the bedside deployment convenience and eliminating the need to transport critically ill patients to dedicated interventional radiology suites equipped with fixed fluoroscopy systems.

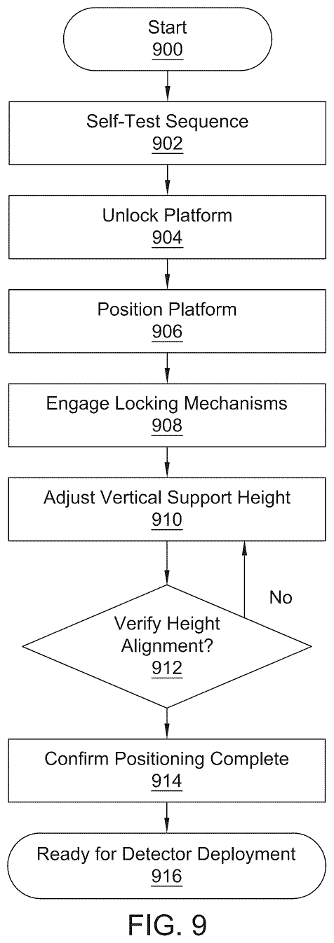

Referring to FIG. 9, a flowchart illustrates the system initialization and positioning sequence performed when setting up the bedside X-ray imaging system for use with a patient. The sequence begins at start block 900 when the system is powered on. The system first executes self-test sequence 902 comprising diagnostic checks of system components to verify operational readiness. Self-test sequence 902 includes checking battery charge level to ensure sufficient power is available for the intended imaging session, verifying motor function by commanding small test movements of the belt drive motor and height adjustment actuators and confirming proper response, and testing position sensors and other feedback devices to ensure accurate position reporting. If self-test sequence 902 detects any fault conditions such as low battery voltage below a minimum threshold, motor malfunction, or sensor failure, the system generates an alert and may inhibit further operation until the fault is corrected.

In some embodiments, the platform is powered by a rechargeable battery system, such as a lithium-ion pack of about 48 V to about 60 V and about 600 Wh to about 1,000 Wh capacity, providing at least about eight hours of typical mixed use. Charging power can be about 500 W to about 800 W with a time to 90 percent state of charge of not more than about 2.5 hours. The power architecture can provide isolated domains for traction, actuation, computation, and detector loads, satisfy medical electrical safety limits for leakage current, and include inrush limiting and power-path management that inhibits charging during exposure.

Upon successful completion of self-test sequence 902, the flowchart proceeds to unlock platform step 904 wherein the operator releases the locking mechanisms if they were engaged during storage or transport. This disengages brakes on the wheels, allowing free mobility of the mobile platform. The flowchart then proceeds to position platform step 906 wherein the operator physically maneuvers the mobile platform on the wheels to position the system adjacent to the patient bed. The operator pushes or pulls the mobile platform, navigating through doorways and around obstacles as needed, until the mobile platform is positioned alongside the patient bed with the vertical support structures aligned with the desired deployment location. The specific positioning depends on the anatomy to be imaged and the side of the bed from which access is available.

In one example aligned with the flowcharts of FIGS. 9 through 11, the method includes step 906 of positioning the mobile platform adjacent to a patient support; step 910 of adjusting height to align a conveyor datum with the patient support surface; step 922 of deploying a detector housing along a longitudinal path defined by the conveyor; verifying alignment criteria; acquiring an image while the detector remains in the extended position; and steps 952 through 968 to retract the detector along the longitudinal path. Interlocks can enforce completion of step 910 before step 922 and completion of retraction before releasing platform brakes.

After achieving the desired lateral position adjacent to the patient bed, the flowchart proceeds to engage locking mechanisms step 908 wherein the operator activates the locking mechanisms. This engages brakes on the wheels or deploys stabilizing feet, immobilizing the mobile platform and preventing unintended movement during subsequent height adjustment and detector deployment operations. Proper locking is verified by the operator observing that the mobile platform does not move when pushed, or by the system monitoring lock engagement sensors and displaying a confirmation message.

With the mobile platform locked in position, the flowchart proceeds to adjust vertical support height step 910 wherein the operator commands height adjustment actuators to extend or retract the vertical support structures until the conveyor belt mechanism is aligned at the height of the patient support surface. The operator actuates height adjustment controls to extend the vertical support structures upward or retract the vertical support structures downward. The operator monitors the height of the conveyor belt mechanism relative to the patient support surface, either by visual observation or by using height indicator displays if provided, and continues adjustment until the deployment path of the conveyor belt mechanism is parallel to the patient support surface. In embodiments with dual vertical columns providing independent height adjustment, the operator adjusts each column separately as needed to accommodate tilted surfaces, ensuring parallelism between the deployment path and patient support surface even when the surface is not horizontal.

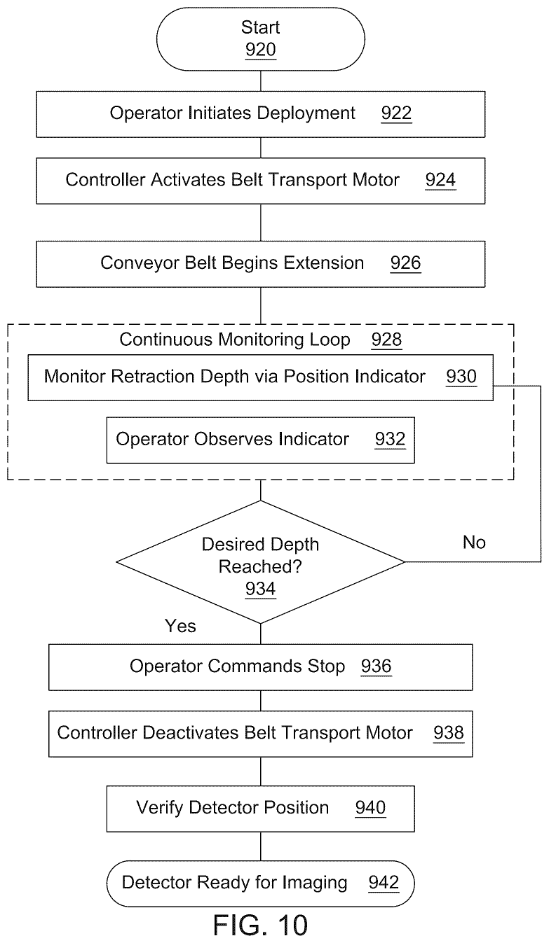

The flowchart includes verify height alignment decision block 912 wherein the system or operator confirms that the height adjustment has been completed satisfactorily. If alignment is not yet satisfactory, as indicated by the “No” path from decision block 912, the flowchart loops back to adjust vertical support height step 910 for additional adjustment. If alignment is satisfactory, as indicated by the “Yes” path from decision block 912, the flowchart proceeds to confirm positioning complete step 914. At this point, the mobile platform is locked in place adjacent to the patient bed, and the conveyor belt mechanism is at the proper height and orientation for deployment beneath the patient. The system displays a ready status message indicating that the system is prepared for detector deployment, and the flowchart proceeds to ready for detector deployment end block 916. The initialization and positioning sequence is now complete, and the operator may proceed with the detector deployment sequence described in connection with FIG. 10.

Referring to FIG. 10, a flowchart illustrates the detector deployment sequence for extending the detector housing assembly from the retracted position to the extended position beneath the patient. The sequence begins at start block 920, which follows from the ready for detector deployment end block 916 of FIG. 9. The flowchart assumes that the system has been successfully initialized and positioned adjacent to the patient bed with the conveyor belt mechanism at the proper height.

The sequence proceeds to operator initiates deployment step 922 wherein the operator commands the start of belt extension. This action signals the controller to activate the belt drive motor. The flowchart then proceeds to controller activates belt transport motor step 924 wherein the controller energizes the belt drive motor, applying electrical power to cause the motor to rotate the drive roller in the forward direction. The belt drive motor may be a DC motor receiving a pulse-width modulated drive signal, a stepper motor receiving step pulses, or a servo motor receiving motion commands via a communication interface.

As the drive roller rotates, the flowchart proceeds to conveyor belt begins extension step 926 wherein the conveyor belt advances in the deployment direction, carrying the detector housing assembly away from the vertical support structures toward the patient bed. The extension continues as long as the belt drive motor remains energized and no stop conditions are detected.

During belt extension, the flowchart enters continuous monitoring loop 928 wherein the system and operator continuously monitor the deployment depth of the detector housing assembly. Within continuous monitoring loop 928, the flowchart performs monitor deployment depth via position indicator step 930. This monitoring may occur through either or both of two mechanisms. In a first mechanism, the operator visually observes position markings on the conveyor belt as they move past a reference indicator, reading the numerical indicia on the position markings to determine how far the detector housing assembly has been deployed. In a second mechanism, the operator observes a deployment depth indicator on a display screen, which presents the deployment distance calculated from position sensor data. The position indicator mechanisms provide real-time feedback to the operator regarding the current deployment depth, enabling the operator to control the deployment process to achieve the desired detector positioning.

Concurrently with monitoring deployment depth, the flowchart performs operator observes indicator step 932 wherein the operator actively watches the position markings or display screen and makes mental note of the current deployment distance. The operator compares the observed deployment depth against the target deployment depth determined based on patient size, anatomy of interest, and other factors.

The flowchart then proceeds to desired depth reached decision block 934 wherein the operator determines whether the detector housing assembly has reached the desired position beneath the patient. If the desired depth has not yet been reached, as indicated by the “No” path from decision block 934, the flowchart loops back to continue monitoring within continuous monitoring loop 928. If the desired depth has been reached, as indicated by the “Yes” path from decision block 934, the flowchart proceeds to operator commands stop step 936 wherein the operator issues a stop command. This stop command may be issued by releasing a button that was held during extension, pressing a dedicated stop button, or using other control input methods.

Upon receiving the stop command, the flowchart proceeds to controller deactivates belt transport motor step 938 wherein the controller removes electrical power from the belt drive motor, causing the motor to stop rotating. The conveyor belt ceases advancing, and the detector housing assembly remains stationary at its current deployed position beneath the patient.

The flowchart then proceeds to verify detector position step 940 wherein the operator visually confirms that the detector housing assembly is properly positioned under the anatomical region of interest. The operator may observe the position markings showing the deployment depth, assess the alignment of the detector housing assembly relative to the patient's body, or use other verification methods. If position verification fails, the operator may repeat the deployment sequence or make fine adjustments using planar displacement mechanisms if provided.

After position verification, the flowchart proceeds to detector ready for imaging end block 942. At this point, the detector housing assembly is deployed beneath the patient at the desired location, and the system is ready for X-ray image acquisition. The operator may proceed to coordinate with the X-ray source operator to acquire images, and after imaging is complete, the operator may retract the detector housing assembly by reversing the belt transport direction through a retraction sequence.

The deployment sequence illustrated in FIG. 10 provides controlled, operator-supervised positioning of the detector housing assembly beneath the patient using the conveyor belt transport mechanism. The continuous monitoring of deployment depth via position indicators enables the operator to achieve accurate positioning without requiring manual manipulation of the patient or detector, reducing physical strain on healthcare staff and improving patient comfort during bedside radiographic procedures.

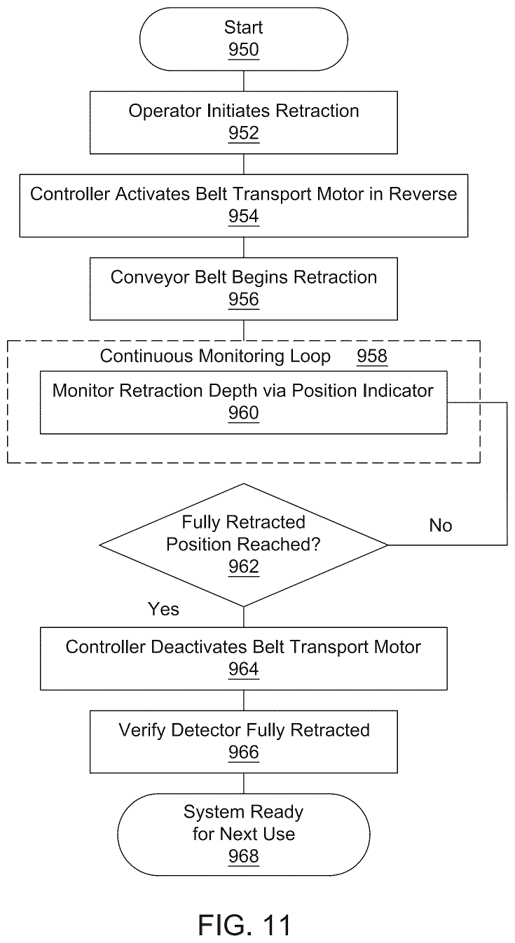

Referring to FIG. 11, a flowchart illustrates the detector retraction sequence for withdrawing the detector housing assembly from beneath the patient back to the retracted position adjacent to the vertical support structures. The sequence begins at start block 950 after imaging procedures have been completed with the detector housing assembly in the extended position beneath the patient.

The sequence proceeds to operator initiates retraction step 952 wherein the operator commands the start of belt retraction. This action signals the controller to activate the belt drive motor in the reverse direction. The flowchart then proceeds to controller activates belt transport motor in reverse step 954 wherein the controller energizes the belt drive motor, applying electrical power to cause the motor to rotate the drive roller in the reverse direction opposite to the deployment direction.

As the drive roller rotates in reverse, the flowchart proceeds to conveyor belt begins retraction step 956 wherein the conveyor belt advances in the retraction direction, withdrawing the detector housing assembly from beneath the patient toward the vertical support structures. The retraction continues as long as the belt drive motor remains energized in reverse and no stop conditions are detected.

During belt retraction, the flowchart enters continuous monitoring loop 958 wherein the system and operator continuously monitor the retraction progress of the detector housing assembly. Within continuous monitoring loop 958, the flowchart performs monitor retraction depth via position indicator step 960. This monitoring occurs through the same mechanisms used during deployment, with the operator observing position markings on the conveyor belt or observing a deployment depth indicator on a display screen showing the decreasing deployment distance as the detector housing assembly withdraws.

The flowchart then proceeds to fully retracted position reached decision block 962 wherein the system or operator determines whether the detector housing assembly has returned to the fully retracted position adjacent to the vertical support structures. This determination may be made by observing that the position indicator shows zero deployment depth, by detecting that the detector housing assembly has reached a mechanical stop or home position, or by sensing the retracted position using limit switches or other position sensors. If the fully retracted position has not yet been reached, as indicated by the “No” path from decision block 962, the flowchart loops back to continue monitoring within continuous monitoring loop 958. If the fully retracted position has been reached, as indicated by the “Yes” path from decision block 962, the flowchart proceeds to controller deactivates belt transport motor step 964 wherein the controller removes electrical power from the belt drive motor, causing the motor to stop.

After motor deactivation, the flowchart proceeds to verify detector fully retracted step 966 wherein the operator or system confirms that the detector housing assembly is fully withdrawn and clear of the patient bed. This verification ensures that the detector housing assembly will not interfere with patient movement or bed adjustments and that the system is ready for transport or storage.

The flowchart then proceeds to system ready for next use end block 968. At this point, the detector housing assembly is retracted, the system may be unlocked and moved away from the patient bed, and the system is ready for transport to the next imaging location or for storage until needed for subsequent imaging procedures.

Safety logic implemented in the controller system monitors for fault conditions and unsafe states, inhibiting operation or generating alerts when such conditions are detected. Safety interlocks prevent belt transport actuation when locking mechanisms are not engaged, preventing detector deployment from an unstable platform. Emergency stop logic responds to activation of an emergency stop control by immediately halting all motor operation and placing the system in a safe state requiring deliberate reset before resuming operation.

In some embodiments, structural elements can be formed from aluminum alloys such as 6xxx series for strength-to-weight efficiency, with low-friction wear surfaces implemented in engineered polymers. Patient-contacting materials can be selected for biocompatibility in accordance with ISO 10993 and for resistance to common hospital disinfectants, including alcohols, quaternary ammonium compounds, bleach solutions, and peracetic acid, with defined surface roughness limits to reduce soil retention. Service access can be provided by a sealed panel with captive fasteners and keyed electrical connectors, enabling replacement of a belt cassette or detector module with a target mean time to repair of about 30 minutes.

The system described herein may be implemented with various alternative embodiments and variations within the scope of the invention. Alternative belt transport mechanisms may include roller-based transport systems wherein the detector housing assembly is supported on rollers that traverse along guide rails rather than being carried by a continuous belt, track-and-carriage systems wherein the detector housing assembly is mounted to a carriage that slides along a track driven by a linear actuator, or cable-driven systems wherein the detector housing assembly is attached to a cable wound around a motorized capstan. Alternative height adjustment mechanisms may include scissor lift assemblies comprising crossed linkages that expand and contract to raise and lower the conveyor belt mechanism, articulated arm assemblies with powered joints providing multi-axis positioning capability, or linear slide systems with motorized ball screws or belt drives providing vertical translation.

Alternative planar displacement mechanism embodiments may include belt and pulley systems, linear motor stages utilizing electromagnetic forces to directly drive the detector mounting platform without mechanical transmission elements, rack and pinion translation mechanisms, piezoelectric actuators providing extremely high resolution positioning through dimensional changes in piezoelectric materials under applied voltage, or electromagnetic positioning stages utilizing magnetic levitation or voice coil actuators providing contactless positioning. Each of these alternative mechanisms provides the functional capability of translating the detector within a two-dimensional plane, differing in implementation details such as resolution, speed, force capacity, and complexity.

Alternative position indicator implementations may include linear scales with visible graduations mounted alongside the conveyor belt, LED or LCD numerical displays integrated into the conveyor belt mechanism showing deployment distance in illuminated digits, graphical displays presenting iconic or pictorial representations of detector position relative to patient anatomy, audible feedback systems wherein the system generates tones or verbal announcements indicating deployment milestones, or tactile feedback mechanisms providing vibration or force feedback to an operator's hand during manual deployment control. The function of the position indicator is to provide the operator with real-time awareness of the deployment depth of the detector housing assembly, enabling the operator to monitor and control the positioning process. This position monitoring function is distinct from patient position detection found in prior art systems, as the present position indicator displays the extension distance of the belt transport system rather than detecting the location of the patient's body.

The locking mechanisms may comprise foot-operated pedal brakes acting on individual wheels, hand-operated lever brakes, electrically activated electromagnetic brakes, central locking systems wherein a single actuator simultaneously locks multiple wheels, or deployable stabilizing elements such as extendable feet, jacks, or outriggers that lower from the platform base to contact the floor and provide additional support points. The function of the locking mechanisms is to immobilize the mobile platform during detector deployment operations, preventing unintended movement that could compromise positioning accuracy or create safety hazards.

The vertical support structures may be implemented as a single central column, dual columns with a connecting arm as illustrated, or multiple columns arranged in other configurations such as a tripod or four-post arrangement. Height adjustment may be manual with hand cranks or handles actuating mechanical advantage systems such as screw jacks or lever mechanisms, semi-automated with power-assisted systems wherein the operator controls the direction of movement but an actuator provides the force, or fully automated with motorized actuators and programmed height control responding to bed height sensing or operator input of target height values.

The wireless communication module may implement various communication protocols including Wi-Fi, Bluetooth, Bluetooth Low Energy, ZigBee, proprietary radio frequency protocols, or cellular data protocols. In certain embodiments, communication may be wired rather than wireless, utilizing cables connecting the system to an X-ray source or to a hospital network. Communication functionality enables optional features such as coordinate system calibration, alignment feedback, remote control, and integration with hospital information systems, but is not required for the core detector deployment functionality which operates based on position indicators and operator control.

The X-ray detector housed within the detector housing assembly may comprise various detector technologies including digital radiography detectors based on amorphous silicon thin-film transistor arrays, direct conversion detectors utilizing materials such as amorphous selenium, indirect conversion detectors utilizing scintillator materials coupled to photodiodes, CMOS-based detectors, or computed radiography cassettes. For interventional applications, CMOS-based detectors provide high frame rates of 30 fps or greater, enabling dynamic tracking of interventional tools such as catheters, though CMOS detectors have increased thickness compared to conventional flat-panel detectors. Conventional digital radiography detectors now also support frame rates of approximately 30 fps in certain implementations. Smaller detector formats may be employed for interventional procedures while maintaining the same detector housing envelope dimensions, with planar displacement providing field-of-view coverage equivalent to larger stationary detectors.

Power supply may utilize lithium-ion battery packs, lithium-polymer batteries, nickel-metal hydride batteries, or other rechargeable battery technologies for portable operation, or may receive power from AC mains via a power adapter or internal power supply converting wall outlet AC voltage to system DC voltages. Hybrid power systems may combine battery power with external power, automatically switching between sources or operating from external power while charging batteries. Power management circuitry within the controller system monitors battery state of charge, manages charging cycles, and implements low-power modes during idle periods to extend battery life.

Cloud-based interface capabilities enable remote management of the system through secure network connectivity. The controller system establishes encrypted communication sessions with cloud-based servers for diagnostics, preventive maintenance, and software updates. Remote diagnostics allow technical support personnel to access system status information, error logs, and performance metrics without requiring on-site presence, reducing response time for troubleshooting and service calls. Preventive maintenance scheduling utilizes usage data and component wear tracking to predict service intervals and automatically notify maintenance staff when service actions are due. Over-the-air software updates deliver firmware updates, feature enhancements, and security patches to deployed systems, ensuring that all units maintain current software versions without manual intervention. The cloud-based interface implements role-based access control, audit logging, and compliance with healthcare data security standards including HIPAA requirements for protected health information.

Smart bed integration capabilities enable the system to interface with patient support structures equipped with IoT positioning sensors or communication capabilities. The system communicates with smart beds via wireless protocols to receive real-time data regarding bed height, tilt angle, and patient positioning. IoT positioning sensors, including RF sensors, provide spatial relationship data between the mobile platform and the patient support surface, enabling the controller to automatically adjust vertical support structure height and angular orientation to match detected bed configurations. For standard beds lacking integrated IoT capabilities, accessory sensors can be attached to bed structures to provide equivalent positioning data. The bed integration functionality enables the system to maintain calibrated spatial awareness of its position relative to the patient support surface throughout positioning and deployment operations, compensating for bed adjustments that occur after initial system positioning. This awareness supports automated height adjustment, tilt compensation, and collision avoidance during detector deployment and retraction.

Autonomous navigation capabilities enable the mobile platform to move within hospital environments with reduced operator intervention. In assisted navigation mode, the system provides powered propulsion while an operator guides the platform using driving handle 106, with motorized wheel drive reducing the physical effort required for maneuvering. In autonomous navigation mode, the system navigates independently to predetermined locations using stored maps, learned pathways, or real-time localization and mapping algorithms. Navigation sensors including cameras, lidar, ultrasonic sensors, or RF positioning beacons provide environmental awareness and obstacle detection during autonomous movement. The autonomous navigation system integrates with hospital information systems to access radiology information system (RIS) data identifying patients scheduled for imaging procedures, automatically routing the mobile platform to designated patient locations to optimize availability and reduce deployment time. Collision avoidance logic halts autonomous movement when obstacles are detected in the travel path, and operator override controls enable manual intervention at any time during autonomous navigation.

Inching capability and position memory enable the system to reproduce positioning from prior imaging sessions for the same patient location. The system stores spatial position data relative to fixed reference points in patient rooms, including distance and angular orientation from walls, doors, or other permanent structures. When returning to a previously imaged patient location, the operator commands the system to recall the stored position, and the system provides guidance or automatically positions itself to match the stored coordinates. Inching controls allow fine positional adjustments in small increments, enabling precise alignment with the recalled position. Position memory reduces setup time for follow-up imaging procedures by eliminating the need to manually determine optimal platform placement, ensuring consistent detector positioning across multiple imaging sessions. The stored position data includes mobile platform location, vertical support structure height, connecting arm extension, and detector housing deployment depth, providing complete configuration recall. Position memory data is associated with patient identifiers or room locations, enabling the system to automatically retrieve appropriate positioning parameters when scheduled imaging procedures are initiated.

The user interface may be implemented with physical buttons, touchscreen displays, voice command recognition, gesture recognition via cameras or motion sensors, remote control devices communicating wirelessly with the controller system, or interfaces on mobile devices such as smartphones or tablets running application software that connects to the system via Wi-Fi or Bluetooth. Multiple interface modalities may be provided simultaneously, allowing operators to choose their preferred interaction method based on circumstances and personal preference.

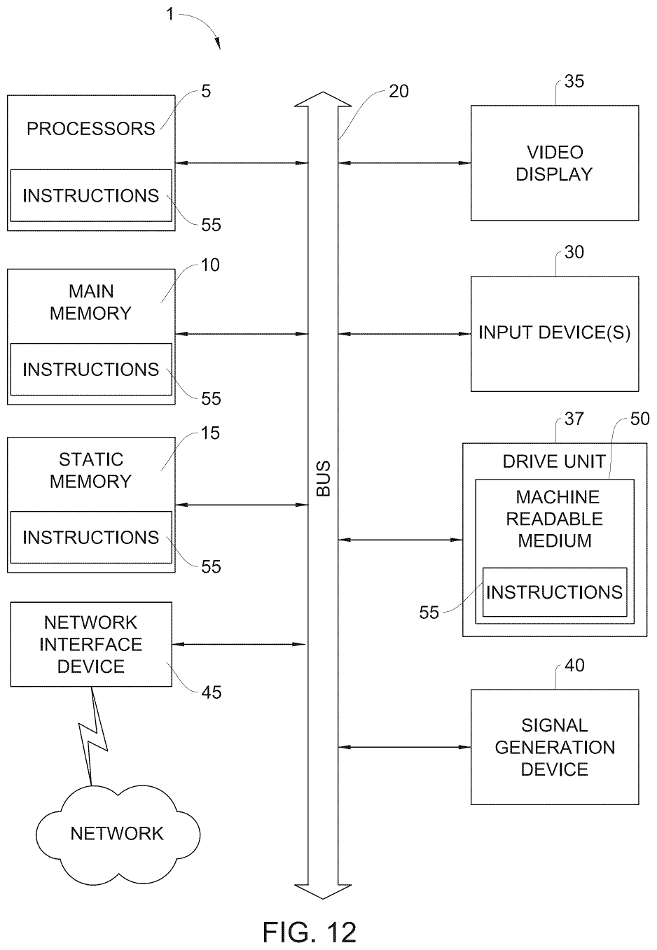

FIG. 12 is a diagrammatic representation of an example machine in the form of a computer system 1, within which a set of instructions for causing the machine to perform any one or more of the methodologies discussed herein may be executed. In various example embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a personal digital assistant (PDA), a cellular telephone, a portable music player (e.g., a portable hard drive audio device such as a Moving Picture Experts Group Audio Layer 3 (MP3) player), a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 1 includes a processor or multiple processor(s) 5 (e.g., a central processing unit (CPU), a graphics processing unit (GPU), or both), and a main memory 10 and static memory 15, which communicate with each other via a bus 20. The computer system 1 may further include a video display 35 (e.g., a liquid crystal display (LCD)). The computer system 1 may also include an alpha-numeric input device(s) 30 (e.g., a keyboard), a cursor control device (e.g., a mouse), a voice recognition or biometric verification unit (not shown), a drive unit 37 (also referred to as disk drive unit), a signal generation device 40 (e.g., a speaker), and a network interface device 45. The computer system 1 may further include a data encryption module (not shown) to encrypt data.

The drive unit 37 includes a computer or machine-readable medium 50 on which is stored one or more sets of instructions and data structures (e.g., instructions 55) embodying or utilizing any one or more of the methodologies or functions described herein. The instructions 55 may also reside, completely or at least partially, within the main memory 10 and/or within the processor(s) 5 during execution thereof by the computer system 1. The main memory 10 and the processor(s) 5 may also constitute machine-readable media.

The instructions 55 may further be transmitted or received over a network via the network interface device 45 utilizing any one of a number of well-known transfer protocols (e.g., Hyper Text Transfer Protocol (HTTP)). While the machine-readable medium 50 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

What is claimed is:

1. A bedside X-ray imaging system, comprising:
a mobile platform comprising a wheeled base and a locking mechanism, wherein the locking mechanism stabilizes the mobile platform adjacent to a patient bed and wherein the mobile platform is structurally independent of the patient bed;
a vertical support structure coupled to the mobile platform, the vertical support structure comprising a height adjustment mechanism that positions a distal portion of the vertical support structure at a height corresponding to and aligned with a patient support surface of the patient bed;
a conveyor belt mechanism coupled to the vertical support structure and configured to extend from the vertical support structure in an orientation aligned and parallel to the patient support surface, the conveyor belt mechanism comprising:
a belt transport system that translates longitudinally relative to the vertical support structure to deploy from a retracted position to an extended position,
a detector housing integrated with the belt transport system, wherein the detector housing travels with the belt transport system during longitudinal translation and houses an X-ray detector, and
a controller operatively coupled to the conveyor belt mechanism, wherein the controller actuates the belt transport system to deploy the detector housing from the retracted position to the extended position beneath a patient positioned on the patient support surface.

2. The system of claim 1, wherein the position indicator comprises visible markings on the belt transport system, the visible markings positioned to be observable by an operator during deployment.

3. The system of claim 1, wherein the position indicator comprises a display panel operatively coupled to the controller, the display panel presenting numerical or graphical representation of detector housing position.

4. The system of claim 1, further comprising a communication interface operatively coupled to the X-ray detector, wherein the communication interface establishes a calibrated coordinate system relating a spatial position of the X-ray detector to a spatial position of a mobile X-ray source positioned external to the mobile platform, and wherein the controller provides feedback regarding alignment between the X-ray detector and the mobile X-ray source based on the calibrated coordinate system.

5. The system of claim 4, wherein the communication interface implements at least one of Wi-Fi or Bluetooth communication protocols.

6. The system of claim 1, wherein the vertical support structure comprises two vertical columns joined by a telescoping horizontal arm, each vertical column comprising an independent height adjustment mechanism enabling differential height adjustment to accommodate a tilted patient support surface.

7. The system of claim 1, wherein the detector housing further comprises a planar displacement mechanism configured to adjust a position of the X-ray detector in two orthogonal directions within a planar envelope defined by the detector housing after the conveyor belt mechanism deploys the detector housing to the extended position beneath the patient, wherein planar displacement occurs within the detector housing transported by the conveyor belt mechanism and is distinct from longitudinal translation provided by the conveyor belt mechanism.

8. The system of claim 7, wherein the planar displacement mechanism comprises actuator assemblies configured to translate the X-ray detector along orthogonal axes, and wherein each actuator assembly comprises at least one of: a lead screw actuator, a belt and pulley system, a linear motor stage, a rack and pinion translation mechanism, a piezoelectric actuator, or an electromagnetic positioning stage.

9. The system of claim 7, wherein the controller is further configured to continuously reposition the X-ray detector via the planar displacement mechanism during an interventional procedure to maintain an interventional device within a field of view of the X-ray detector as the interventional device advances through patient anatomy, and to generate a composite X-ray image by mosaicking multiple X-ray images acquired at different detector positions based on position data from the planar displacement mechanism.

10. A bedside X-ray imaging system comprising:
a mobile platform positionable adjacent to a patient bed and structurally independent of the patient bed, the mobile platform comprising a wheeled base and a locking mechanism;
a conveyor belt mechanism coupled to the mobile platform and configured to extend from the mobile platform toward the patient bed in an orientation parallel to a patient support surface, the conveyor belt mechanism comprising:
a detector housing that houses an X-ray detector, wherein the detector housing translates with the conveyor belt mechanism from a retracted position to an extended position beneath a patient positioned on the patient support surface, and
a position indicator that displays a deployment depth of the detector housing during translation; and
a controller operatively coupled to the conveyor belt mechanism, wherein the controller actuates the conveyor belt mechanism to insert the detector housing beneath the patient from a bedside position without requiring mechanical coupling to the patient bed.

11. The system of claim 10, further comprising a vertical support structure coupling the conveyor belt mechanism to the mobile platform, the vertical support structure comprising a height adjustment mechanism.

12. The system of claim 10, wherein the detector housing comprises a planar displacement mechanism that adjusts a position of the X-ray detector in two orthogonal directions within an envelope of the detector housing after deployment to the extended position.

13. The system of claim 10, further comprising proximity sensors coupled to the conveyor belt mechanism, wherein the proximity sensors detect contact between the conveyor belt mechanism and the patient bed.

14. The system of claim 10, wherein the detector housing comprises a service access feature configured to enable installation or replacement of the X-ray detector during factory configuration or field service procedures, and wherein the detector housing is sealed during operational deployment to prevent routine operator access to the X-ray detector.

15. A method of performing bedside X-ray imaging, the method comprising:
positioning a mobile X-ray detector deployment device adjacent to a patient bed, wherein the mobile X-ray detector deployment device is structurally independent of the patient bed and comprises a conveyor mechanism housing an X-ray detector;
adjusting a height of the mobile X-ray detector deployment device to align the conveyor mechanism with a patient support surface of the patient bed;
actuating the conveyor mechanism to deploy the X-ray detector from a retracted position external to the patient bed to an extended position beneath a patient on the patient support surface, wherein the X-ray detector translates along a path defined by the conveyor mechanism during deployment;
monitoring a deployment depth of the X-ray detector during actuation using a position indicator on the conveyor mechanism; and
acquiring an X-ray image of the patient using the X-ray detector while the X-ray detector remains in the extended position beneath the patient.

16. The method of claim 15, wherein monitoring the deployment depth comprises visually observing markings on the conveyor mechanism indicating longitudinal displacement.

17. The method of claim 15, wherein monitoring the deployment depth comprises viewing a display panel presenting a representation of detector position relative to the patient.

18. The method of claim 15, further comprising:
establishing a calibrated coordinate frame relating a spatial position of the X-ray detector to a spatial position of a mobile X-ray source positioned external to the mobile X-ray detector deployment device; and
determining, based on the calibrated coordinate frame, whether an alignment criterion between the X-ray detector and the mobile X-ray source is satisfied prior to acquiring the X-ray image.

19. The method of claim 15, further comprising adjusting a planar position of the X-ray detector within a detector housing after deployment to the extended position and prior to acquiring the X-ray image, wherein adjusting the planar position occurs within an envelope of the detector housing and is distinct from longitudinal translation provided by the conveyor mechanism.

20. The method of claim 19, further comprising continuously repositioning the X-ray detector during an interventional procedure to maintain an interventional device within a field of view of the X-ray detector, acquiring multiple X-ray images at different detector positions, and generating a composite X-ray image by mosaicking the multiple X-ray images based on position data corresponding to the different detector positions.
